(19) United States
(12) Patent Application Publication
(10) Pub. No.: US 2006/0073540 A1
(43) Pub. Date: Apr. 6, 2006

(54) Method and system for controlling micro-objects or micro-particles

(75) Inventor: Sylvain Martel, Newton, MA (US)

Correspondence Address: VENABLE LLP, P.O. BOX 34385, WASHINGTON, DC, 20045-9998, US

(73) Assignee: LA CORPORATION DE L'ECOLE POLYTECHNIQUE DE MONTREAL, Montreal (CA)

(21) Appl. No.: 11/145,007

(22) Filed: Jun. 6, 2005

Related U.S. Application Data
(60) Provisional application No. 60/576,609, filed on Jun. 4, 2004.

(51) Int. Cl. C12Q 1/04; C12N 1/20

(52) U.S. Cl. 435/34; 435/252.1

(57) ABSTRACT

Moving components with dimensions much smaller than what micro-electro-mechanical system (MEMS) technology can accomplish in terms of force generated and power efficiency, are integrated onto micro-systems. These moving components referred to herein as “bio-components” are special bacteria (magnetotactic bacteria) where the motion of these bio-components can be controlled by generating an “artificial pole” by a magnetic field generated by a constant electrical current that can be varied to change the location of the “artificial pole” from an electrical system such as an embedded electronic micro-circuit. According to the present invention, it is possible through a software program or codes, to control the direction of motion of these magnetotactic bacteria, for example, by downloading such program onto the embedded electronics (controller or the like), and to generate the required magnetic field to control such bacteria to accomplish a particular task. Moreover, though integrated sensory means and new algorithms, the magnetotactic bacteria-based system could adapt or change the direction of motion from new occurring conditions.

### CROSS-REFERENCE TO RELATED APPLICATIONS

Priority is claimed from U.S. Provisional Application No. 60/576,609, filed Jun. 4, 2004, the entire content of which is incorporated herein by reference.

### FIELD OF THE INVENTION

The present invention generally relates to microsystems. More specifically, the present invention is concerned with method and system for controlling micro-objects or micro-particles.

### BACKGROUND OF THE INVENTION

As micro-mechatronic systems including microrobots become smaller, the design and integration of mechanical components, even with the use of silicon micro-electro-mechanical system (MEMS), becomes extremely difficult if not impossible compared to the micro-electronic counterparts when scaled down to a few micrometers.

The ability to manipulate suspended microparticles in liquid has significant potential for applications in Microsystems such as lab-on-a-chip and in micro-total-analysis systems (μTAS) technology. Existing micromanipulation methods such as electro-osmosis [1] and dielectrophoresis [2] (DEP) are based on the principle of electrokinetics where electrical power is required to induce a force using relatively high frequencies and voltage amplitudes that depend on dielectric properties. These methods are most likely to be incompatible with the embedded electronics that tends towards lower operating voltages, resulting in larger devices through added voltage converters with further decrease of the electrical power efficiency.

Bacteria have been previously used effective for many operations in low Reynolds number hydrodynamics [5] as it is the case in microfluidic systems. The integration of bacteria as functional components has been previously done when Serratia marcescens flagellated bacteria were attached to polydimethylsiloxane or polystyrene to form a bacterial carpet for moving fluid or to move a bead or chip at about 5 μm/s when attached to it [6]. In previous examples [6,7], bacteria were acting without external control. Without coordination, not only the propulsion force F generated by N bacteria attached to an object of size L scales like F proportional to N^{1/2} assuming N proportional to L², but the displacement paths result into the so-called run-and-tumble strategy that can be explained by a chemotaxis model [8] while remaining unpredictable. Considering the above, controlling the direction of motion of N bacteria attached to an object of size L is expected to scale like F proportional to N.

Unlike most bacteria that are based on chemotaxis to detect nutrient gradients and hence influence their motility [9-11], the direction of displacement of magnetotactic bacteria (MTB), with their chain of magnetosomes which are membrane-based nanoparticles of a magnetic iron, although influenced by chemotaxis and aerotaxis, is mainly based on magnetotaxis [12-14] which represents a more suitable interface with electronics. The motility of MTB has been exploited in the past through the use of permanent magnets or electromagnets, typically in mass-scale applications such as low field orientation magnetic separation [15] (OMS) being a process in which motile, magnetic field susceptible MTB can be separated. Controlled micromanipulation of MTB using microelectromagnets arrays have also been reported [16-17].

It has also been proposed to use bacteria to create small systems, such as bacteria carpet used to pump fluid in a micro-channel, but without achieving control on these bacteria. Indeed, in this last example, the pumping action is done by attaching bacteria on the walls of the fluidic channels and the flow is created by the uncontrolled movement of their flagella.

### SUMMARY OF THE INVENTION

According to the present invention, there is provided a method for controlling the motion of bacteria through electrical means and to integrate them as components in miniature systems. As micro-mechatronic systems including microrobots become smaller, the design and integration of mechanical components, even with the use of silicon micro-electro-mechanical system (MEMS), becomes extremely difficult if not impossible compared to the micro-electronic counterparts when scaled down to a few micrometers. As such, the present invention allows replacing some of the smallest mechanical components by living organisms and more specifically with bacteria with overall dimensions in the order of a few micrometers to create a new field of micro-biotronic or micro-biomechatronic systems.

More specifically, in accordance to a first aspect of the present invention, there is provided a method for controlling at least one magnetotactic bacterium (MTB), the at least one MTB being self-propulsive along a displacement path, the method comprising: generating a magnetic field so as to effect the at least one MTB; the magnetic field being characterized by a pole; the magnetic field affecting the at least one MTB by biasing the displacement path towards the pole; and selectively modifying the displacement path of the at least one MTB by modifying the pole of the magnetic field.

According to a second aspect of the present invention, there is provided a method for controlling at least one micro-object comprising: providing at least one magnetotactic bacterium (MTB); the at least one magnetotactic bacterium being self-propulsive; coupling the at least one micro-object with the at least one magnetotactic bacterium, causing the at least one micro-object to move in unison with the at least one magnetotactic bacterium; and generating a magnetic field for orienting the at least one magnetotactic bacterium along a displacement path; whereby, in operation, modifying the orientation of the magnetic field allows modifying the displacement path of the at least one magnetotactic bacterium, thereby allowing controlling the path of the at least one micro-object during displacement thereof by the at least one magnetotactic bacterium.

In order to control the motion or displacement of these “bio-components” from an electronic circuit embedded onto the same system, a special type of bacteria known as magnetotactic bacteria is used. Past studies have shown that magnetotactic bacteria migrate in the direction of the Earth magnetic pole that is sensed by the bacteria through a chain of ferromagnetic particles, which are a few tens of nanometers in size, embedded onto the bacteria.

According to the present invention, magnetotactic bacteria, with dimensions much smaller than what MEMS technology or others can accomplish, are therefore integrated as functional components within a microsystem and especially for the controlled manipulation of micro-objects or microparticles.

In accordance to a third aspect of the present invention, there is provided a system for controlling at least one micro-object comprising: at least one magnetotactic bacterium (MTB) for coupling with the at least one micro-object for movement in unison; the at least one magnetotactic bacterium being self-propulsive; and a magnetic field generator for orienting the at least one magnetotactic bacterium along a displacement path; whereby, in operation, modifying the orientation of the magnetic field allows modifying the displacement path of the at least one magnetotactic bacterium, thereby allowing controlling the path of the at least one micro-object during displacement thereof by the at least one magnetotactic bacterium.

The motion of these moving components can be controlled by generating an “artificial pole” by a continuous magnetic field generated by a direct current (DC) electrical signal from an electrical system such as an embedded electronic micro-circuit. No known prior art technologies allows to miniaturize at such a scale with the power efficiency related to the force provided by these bacteria.

The present invention provides also a new controlled micromanipulation and microactuation method independent of the dielectric properties where micro-objects such as microbeads are manipulated and actuated by Magnetospirillum gryphiswaldense bacteria [3] for example. The method can operate, for example, under the influence of computer-based software. This current-driven method minimizes electrical power requirement and hence heat generation by exploiting the motility of magnetotactic bacteria [4] (MTB). Beside microbeads, controlled MTB can also be integrated in microrobots or in microsystems as actuators for switches, micro-valves, pistons, and micro-motors, to name a few.

Advantages of method and system according to the present invention includes: extremely small moving components, very small electrical power with regards to other technologies, relatively large force achieved, several applications such as moving body inside a fluid, etc.

Since the lifetime of the bacteria under these conditions are relatively short, some conditions of their environment are controlled in order to extend their lifetime.

It is to be noted that a system and method according to the present invention allows controlling micro-objects, micro-particles or any other objects or entity small enough to be carried by magnetotactic bacteria. Therefore, even though in the present specification, references are often made in relation to some embodiments specifically to micro-objects for example and in relation to other embodiments to micro-particles for example, those specific embodiments are not restricted to the referred object unless specified.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1 is a flowchart of a method for controlling micro-objects according to an illustrative embodiment of the present invention;

FIG. 2 is a schematic view of a system for controlling micro-objects according to a first illustrative embodiment of the present invention;

FIG. 3 is an image of a system for controlling micro-objects according to a second illustrative embodiment of the present invention;

FIG. 4A are graphs illustrating displacement speeds with and without the presence of an artificial magnetic field showing a Maxwell distribution following motility and response from a sample of 295 and 959 MTB respectively measured by images analysis;

FIG. 4B is a graph illustrating an extrapolation line showing the percentage of the MTB oriented within 60 degrees along the line of an applied magnetic field at various intensities for the sample of FIG. 4A;

FIG. 4C is a graph illustrating the swimming direction of the bacteria from the sample of FIG. 4A without an applied magnetic field;

FIG. 4D is a graph illustrating the swimming direction of the bacteria from the sample of FIG. 4A with an applied field of 3.5 gauss along the x axis; the data being normalized to unity with magnitudes corresponding to the swimming speeds of the MTB;

FIGS. 5A-5F are close view images having edges of 36.0 μm illustrating controlled manipulation of a first microbead being pushed by a magnetotactic bacteria (MTB); FIG. 5A showing chaotic movements of the MTB when no artificial magnetic field is applied; FIG. 5B showing the path of one MTB not attached to a microbead during manipulation; FIG. 5C showing the displacement of one microbead pushed by one MTB with a voluntary change in the direction of the path; FIGS. 5D-5F respectively showing the initial position, the position when directional change is applied and the final position of the manipulated mobile microbead;

FIGS. 6A and 6B are close view images of a second microbead being pushed by a single MTB in a desired direction; FIG. 6A representing 16 sequential frames; and FIG. 6B representing the resulting overlapped image;

FIGS. 7A-7B are images of oscillatory displacement paths of a single microbead pushed by a MTB, respectively illustrating oscillatory trajectory on a 230 μm long path and a oscillatory-linear variation;

FIG. 8 is a schematic view of a bacterial mixer according to an illustrative embodiment of a first application of the method from FIG. 1; and

FIG. 9 is a schematic view of a redundant bacterial micro-system according to an illustrative embodiment of a second application of the method from FIG. 1.

### DETAILED DESCRIPTION

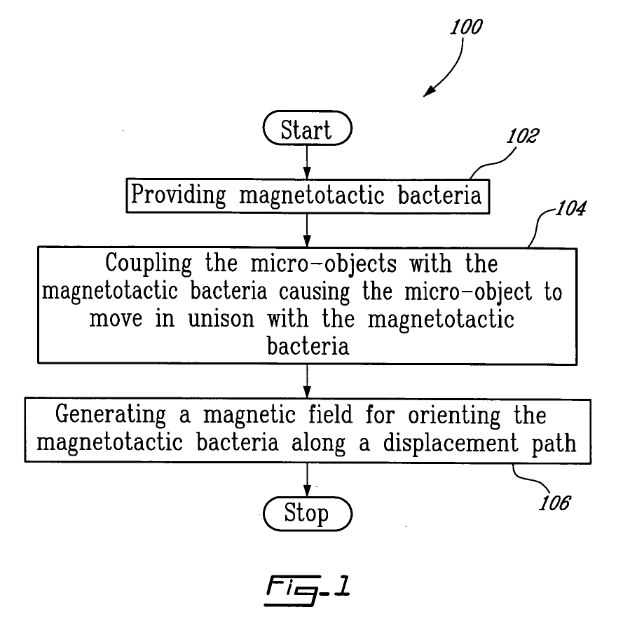

A method 100 for controlling micro-objects or micro-particles according to an illustrated embodiment of the present invention will now be described with reference to FIG. 1.

The method 100 comprises the following steps:

102—providing magnetotactic bacteria;

104—coupling the micro-objects with the magnetotactic bacteria, causing the micro-objects and the magnetotactic bacteria to move in unison; and

106—generating a magnetic field for orienting the magnetotactic bacteria along a displacement path.

As will become apparent upon reading the following description, modifying the orientation of the magnetic field allows modifying the displacement path of the magnetotactic bacteria, thereby allowing controlling the path of the micro-objects during displacement thereof by the magnetotactic bacteria.

Also, as will become apparent hereinafter, steps 104 and 106 may be inverted so that a magnetic filed is first generated so as to orient the magnetotactic bacteria along a displacement path so that they move towards the micro-objects and then couple therewith so that their movement in unison acts upon the micro-objects.

Each of these steps will now be described in more detail.

In step 102, magnetotactic bacteria (MTB) are provided. MTB are of course living organism and self-propulsive. The number of MTB provided may vary depending on the application and on the number or nature of the objects to control. In some cases, as will be illustrated hereinbelow with references to specific examples, a single MTB may be sufficient to move a micro-object or micro-particle.

The MTB are provided, for example, in the form of an active solution of highly concentrated magnetospirillum gryphiswaldense bacteria in a fresh culture medium.

Even though other types of MTB can be used, this particular bacterium has demonstrated the best result in term of synthesis of magnetosomes with a longer chain than the ones typically found in other MTB and hence a faster response to a directional change of a low magnetic field. This long chain of magnetosomes imparts to the MTB a magnetic moment that generates sufficient torque so that the bacteria can align themselves to magnetic field lines as will be explained hereinbelow in more detail with reference to step 106. The chain had up to 50 cubo-octahedral magnetosomes (Fe₃O₄) grown by microaerophilic Magnetospirillum strains and a microaerobic fermentation procedure.

In step 104, the magnetotactic bacteria are fixed to the micro-objects, causing the micro-objects to move in unison with the magnetotactic bacteria.

To enable the MTB to stick to the micro-objects, the MTB are forced to generate activation of lipopolysaccharides. For example, the bacteria are mixed in a medium poor in concentration of nutrients prior to injecting a new concentration in a new medium containing a concentration of micro-objects.

Other means to fix the micro-objects to the MTB are well known in the art of micro-biology and thus will not be described herein in more detail.

As will become more apparent hereinafter, depending on the applications other coupling means can be provided between the MTB and the micro-objects. For example, the MTB may be enclosed in a micro-structure, then acting as the micro-object.

In step 106, once the micro-objects to control are fixed to or more generally coupled with the MTB, a magnetic field is generated for orienting the magnetotactic bacteria along a displacement path. The MTB then simply follows this displacement path, bringing with them the micro-objects they carry.

In the method 100, the motility of the magnetotactic bacteria is used. Magnetotactic bacteria are very efficient organisms and exploiting their energy and motility translates into efficient micro-systems in term of energy required to do a particular task, a real benefice in miniature systems. Unlike other methods of manipulation such as electrophoresis or photon trapping (optical tweezers), the magnetic field is created using a DC signal instead of an AC signal. Also, unlike other methods, the magnetic field is not intended to induce a gradient force on magnetic particles inside the bacteria but to induce a torque to orient the bacteria on an artificial “magnetic north” or the like created by the electrical circuit to change or modify the path of migration of the bacteria. Hence, the mobility and energy of the bacteria is exploited in order to decrease the energy required for the electronics. Furthermore, the method 100 does not include inducing a gradient force through a magnetic field directly on an object but rather use a low energy DC magnetic field, where intensity and orientation are controlled by an electronic system, to direct the motion of the bacteria in order for them to swim using their own energy to a specific location to perform a particular task such as pushing or pulling on an object.

Since the lifetime of the bacteria are relatively short, some conditions of their environment are controlled in order to extend their lifetime as it is commonly known in the art of micro-biology.

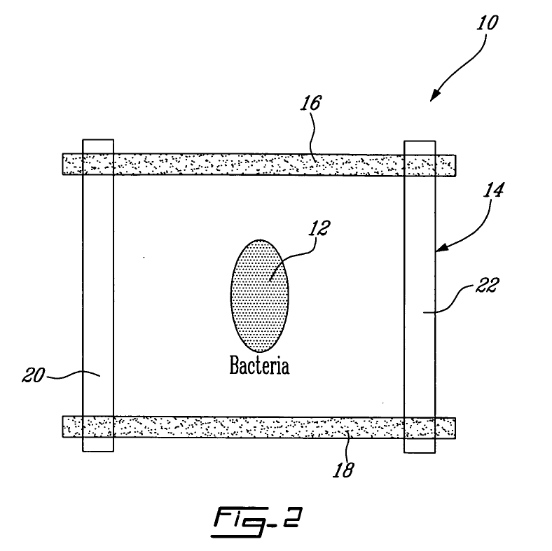

Turning now to FIG. 2 of the appended drawings a system 10 for controlling a micro-object (not shown) according to a first illustrative embodiment of the present invention will now be described.

The system 10 comprises a magnetotactic bacterium (MTB) 12 to receive the micro-object to be controlled and a magnetic field generator 14 for orienting the magnetotactic bacteria along a displacement path as determined by the magnetic field generator 14. As described hereinafter, the micro-object is fixed to the MTB so as to move in unison therewith. It is reminded that no electrical power is required to move the MTB/micro-object ensemble since the MTB is self-propulsive, being a living organism.

The magnetic field generator 14 includes four insulated conductor or micro-wire 16-22, configured in two perpendicular facing pairs defining a generally rectangular shape, for receiving independent electrical signals. In operation, modulating the signal amplitude of wires in pair (such as 16-22, 16-20, 18-20, or 18-22), yields a desired vectored location of an artificial pole, enabling accurate angular bacterial displacement.

Each of the four conductors 16-22 are coupled to a signal controller (not shown). The controller may take many forms from an electric circuit to a computer including output port for connecting the conductors 16-22 and software for controlling the signals in the conductors 16-22 depending on preprogrammed or user input instructions. It is believed to be within the reach of a person skilled in the art to program a computer or conceive a circuit to control the signals in the conductors 16-22. The controller allows positioning the artificial pole at various angles around the bacterium in order to control its direction of displacement.

Under sensory means or visual feedbacks (not shown), such as with the use of a microscope, an input device (not shown) in the form of a joystick, a mouse or of a keyboard for example, connected to the controller, may be used to allow an operator to modify the orientation of the magnetic field produced by the magnetic field generator 14, thereby allowing controlling the path of the micro-object during displacement thereof by the magnetotactic bacteria under the influence of the magnetic field.

Other wire configurations than the illustrated rectangular configuration are also possible depending on the applications. Also, the number of wires may vary.

Examples of sensory means include photovoltaic cells, magnetic sensors, chemical sensors, etc. and depend of course on the application.

The magnetic field generator 14 may also be in the form of a permanent magnet.

The system 10 may include a plurality of MTB 12 allowing to simultaneously controlling a plurality of micro-objects.

Since the electromagnetic field only needs to be slightly larger than the Earth magnetic field when no special shielding is implemented, coped with the fact that the energy for the motion is provided by the bacteria and not by the external field, very small electrical power requirement (current and voltage) is required with the system 10 to enable controlled motion of the micro-object. This low power requirement translates into a significant benefit for extremely small systems compared to other technologies including MEMS.

Alternatively, an embedded computer (controller or the like) can be provided including a software program or codes to control the direction of motion of the MTB to generate the required magnetic field to control such bacteria to accomplish a particular task. Moreover, through integrated sensory means, the magnetotactic bacteria-based system is allowed to adapt or change the direction of motion from new occurring conditions. It is believed that the current state of miniaturization allows for such an embedded computer. It is therefore believed to be within the reach of a person skilled in the art to conceive such an embedded system.

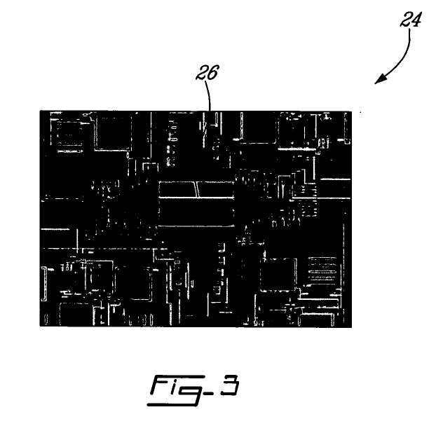

A system 24 for controlling micro-objects (not shown) according to a second illustrative embodiment of the present invention will now be described with reference to FIG. 3.

The system 24 is in the form of a bacterial manipulator allowing for the manipulation of micro-objects or micro-objects connected to other objects such as microbead to DNA strands, and includes a grid 26 of conductors similar to the conductors 16-22 in FIG. 2, where magnetotactic bacteria (MTB) can operate. More specifically, the grid includes an array of at least two sets of conductors in parallel passing at right angle from each other as illustrated in FIG. 3.

Under sensory means or visual feedbacks, such as with the use of a microscope, specific conductors are activated by passing an electrical current such that the direction of motion of the MTB or groups of MTB can be controlled, similarly to what has been described with reference to the operation of the system 10. Several MTB or groups of MTB can be controlled simultaneously and independently to push objects to the desired location. According to a more specific embodiment, an input device (not shown), in the form of a mouse or other devices, is used to point the location where the micro-objects have to be placed. Through imaging, the system 24 controls the MTB to move the object or objects to the desired location by sending the current in selected conductors. It is to be noted that minimizing the space between conductors also allows a decrease of the electrical current required.

The system 24 can be modified to form a “bacterial” display (not shown), where the objects that are moved by the MTB under the influence of controlled localized magnetic field become fragments or pixels to form lettering, numbers, or other shapes in order to display texts or drawings as any displays would do. Of course, such a display includes a controller for coordinating relative displacements of at least some of the micro-objects in unison with respective MTB so as to be moved at selected positions on the grid.

Experimental Results

A first experiment has been conducted where a micro-piston at low Reynolds numbers in a cylinder was pushed and pulled with the aid of magnetotactic bacteria controlled by an electro-magnetic field generated by an electronic system. In this particular experiment, bacteria concentrations of 10⁷ and 10¹⁰/ml were used and displacement velocities of the bacteria between 0 and 200 μm/sec and flow speeds generated by the bacteria between 0 and 50 μm/sec depending on the field applied, were observed. Micro-canals of various dimensions were used (250 μm×100 μm×3 mm; 250 μm×100 μm×1 mm; 250 μm×200 μm×3 mm; 250 μm×200 μm×1 mm; 250 μm×300 μm×3 mm; etc. to evaluate the critical magnetic field for operation in micro-system.

It has also been observed that the time for a 180 degrees rotation of the bacteria takes less then one second with a less than 2 gauss field. Although there are many types of magnetotactic bacteria that could be used, DSMZ 3856 magnetospirillum magnetotacticum active culture and DSMZ 6361 magnetospirillum gryphiswaldense active culture with medium 380 and medium 512 were used for the first experiments.

The micro-piston was fabricated with a glass fiber with a diameter between 30 and 80 μm and covered with PDMS with an opening of the capillaries varying between 150 and 250 μm. The lengths of the fiber pulled by the bacteria under control were between 1 mm and 40 mm.

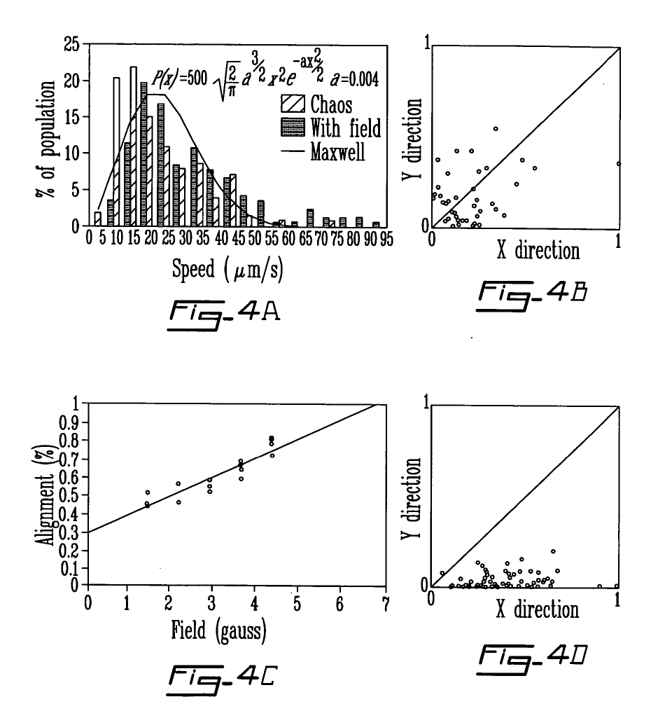

Active solutions of highly concentrated Magnetospirillum gryphiswaldense bacteria were chosen because these particular bacteria demonstrated the best result in term of synthesis of magnetosomes with a longer chain than the ones typically found in other MTB and hence a faster response to a directional change of a low magnetic field. This long chain of magnetosomes imparts to the MTB a magnetic moment that generates sufficient torque so that the bacteria can align themselves to magnetic field lines. The chain had up to 50 cubo-octahedral magnetosomes (Fe₃O₄) grown by microaerophilic Magnetospirillum strains and a microaerobic fermentation procedure [18]. The solution containing the bacteria showing the most motility from observation under a microscope with the best response to magnetic field (see FIGS. 4a-4d) from a permanent magnet was selected and concentrated in a centrifuge to form a condensate.

To force the MTB to generate activation of lipopolysaccharides to allow them to stick to the microbeads, the bacteria were remixed in a medium poor in concentration of nutrients for 5 minutes prior to inject a concentration between 1×10⁶ and 1×10⁸ BMT/ml in a new medium containing an average concentration of 5×10⁶ microbeads/ml. The 3 μm in diameter highly uniformed microbeads from Fluka™ (MDL number MFCD00197912) with a density of 1.51 g/cm³ and made of Melamine-formaldehyde resin show hydrophilic properties and long term stability in an aqueous solution. It has been observed that during the first 5 minutes when placed in the new medium in custom-made microfluidic channels, less than 1% of the bacteria attached themselves to the microbeads and could even push them along the line of an applied magnetic field. To validate that the movement of the microbeads was due entirely by the pushing action of the MTB, dead MTB after being exposed to microwave irradiation and taken from the same culture were added and did not showed any movement. Previous attempts using the MTB directly from the received culture showed that the bacteria would not push the microbeads but rather ignore them.

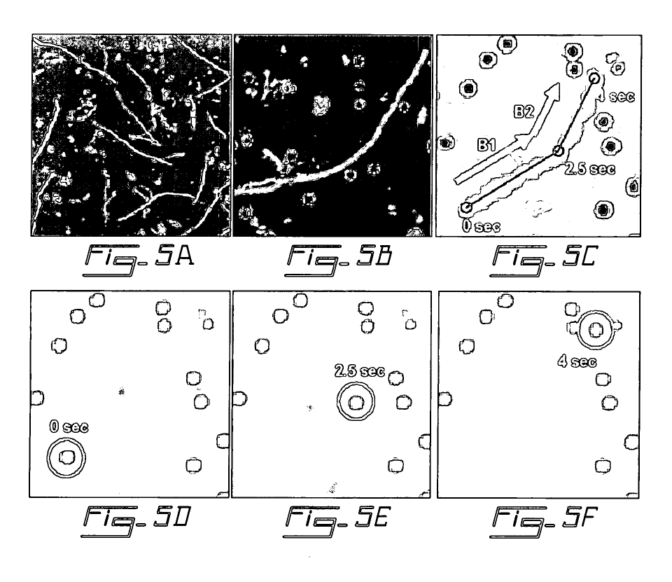

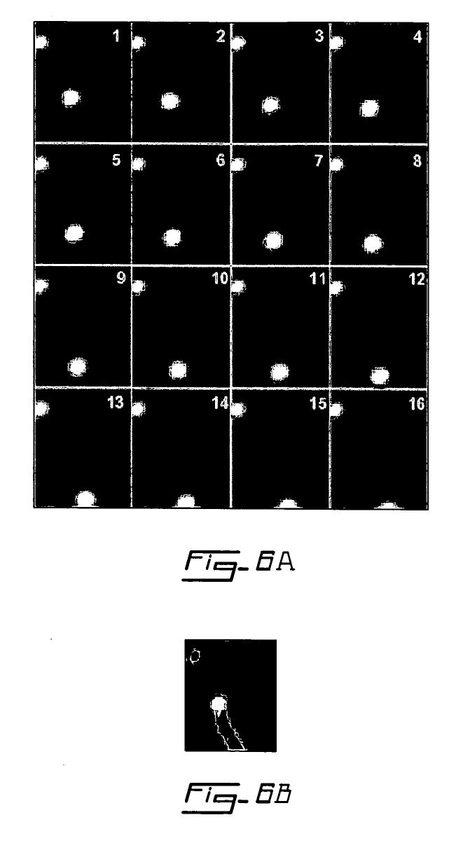

FIGS. 5A-5F illustrates the controlled manipulation of a microbead being pushed by a MTB. As illustrated in FIG. 5A, initially, chaotic displacements were observed when no magnetic field was applied.

A simple example among many successful results, which is illustrated in FIG. 5C, validates the controlled manipulation of microbeads by MTB. In this particular example, the direction of a manipulated microbead was shifted about 300 counterclockwise after about 2.5 seconds. The traces of the movements of the microbead or bacteria that can be seen in FIG. 5C were obtained by the superposition of images taken at different time. The control of the movement of the MTB was performed in some cases using permanent magnets and in other experiments a simple program written in C++ and compiled prior to the experiment was coded to change the direction of the MTB through electromagnetic grids similar to those illustrated in FIG. 3 and which will be described hereinbelow in more detail. The experimental grids were simulated and conceived to achieve a uniform field within the observation pool. A constant magnetic field through the observation pool guaranteed that the magnetic moment of the magnetosomes chain would not interact with the magnetic field gradient, but rather functions as a navigational compass using the general torque [19].

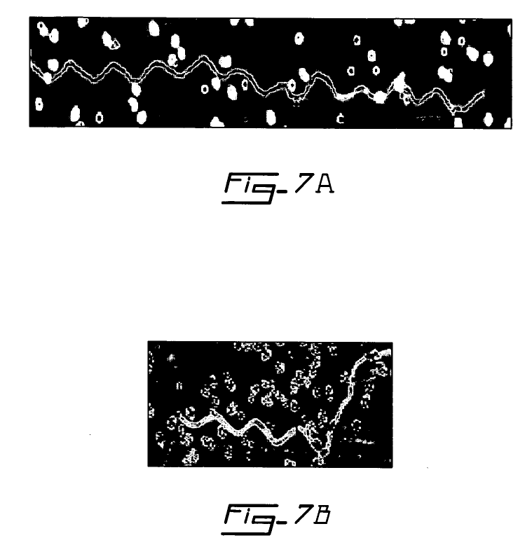

A close-up view (see FIG. 6A) of another microbead being manipulated shows and confirms that the microbead is actually pushed by a single and not by several MTB. The directions of the movements of the mobile microbeads were similar to the movements of MTB not attached to a bead, which is illustrated in FIG. 5b.

A video analysis gave an average speed for a set of microbeads being manipulated by a single MTB (which will be referred to herein as “mobile microbeads”) of 7.5 μm/s with a peak velocity of 20 μm/s. Since the viscous drag on a sphere is proportional to its radius and knowing that the cell body of the MTB is smaller than the diameter of the microbead being pushed, an average speed of 7.5 μm/s and a peak speed of 20 μm/s would correspond to an average and a peak velocity of the MTB without the bead of 22.5 μm/s and 60 μm/s respectively. These values appear to be consistent with the previously measured speeds of the MTB (see FIG. 4A). Even though the recorded average speed of this particular sample of MTB was below typical average speeds of other types of MTB such as Magnetospirillum sp. AMB⁻¹ cells, where an average swimming speed of 49 μm/s with a standard deviation of 20 μm/s, has been recorded [20], it is believed that average velocities comparable to other bacteria could be achieved. The experimental results suggest that a single MTB could move a bead of 3 μm, 10 μm, and 100 μm in diameter with an average speed of about 16.3, 4.9, and 0.49 μm/s respectively, corresponding from Stokes' law, to a thrust of about 0.5 pN per MTB. Furthermore, since the low Reynolds number drag also scales like the size of the object, we can expect higher velocities with more MTB attached to larger objects.

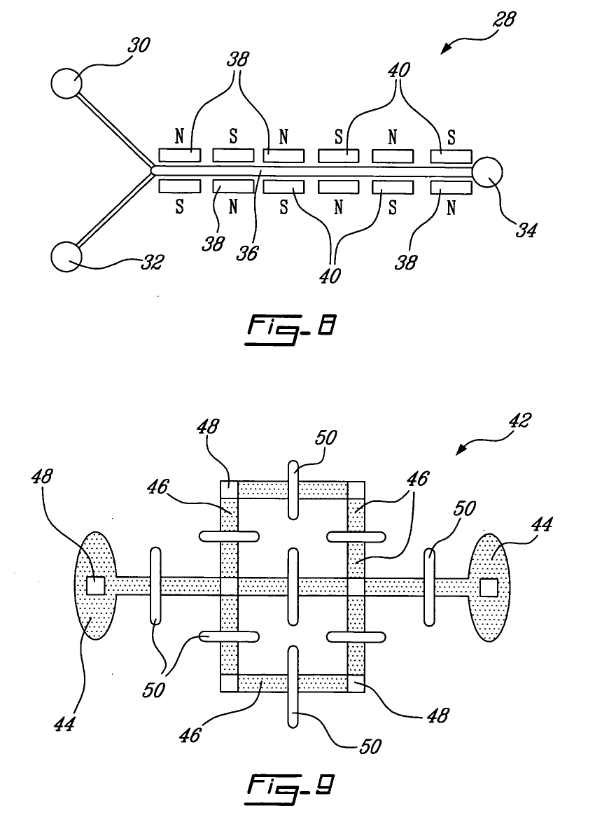

Measurements by video analysis also showed some small variations in velocity of the mobile microbeads. It is believed that these variations in velocity are attributed to some extents to collisions with other microbeads and the effects of neighbor bacteria. For instance, from previous observations [6], one can expect that the speed of a bacterium or a mobile microbead to be affected by any bacteria within a range of about 50 μm ahead of it with a larger probability of occurrences with higher concentrations of bacteria. As illustrated in FIG. 7A, in some cases, the mobile microbead also showed oscillatory displacement paths in the same direction where other mobile microbeads were moving in a straight line and this oscillatory behavior disappeared when the direction of the field was changed (see FIG. 7B).

Based on current-carrying manipulation microcircuits [21] that result into sufficient heat to be problematic for many applications, bacterial manipulation and bacterial actuation may reduce the required current by at least 100 folds while not being restricted to superparamagnetic microbeads. Since MTB can orient themselves and swim along the lines of the geomagnetic field (0.5 gauss) coped with the fact that magnetic moment for MTB reaching 1×10⁻¹⁵ A•m² has been measured [16], suggests that a minimum current amplitude of less than 1 mA or even a few hundreds micro-amperes could be envisioned with a pre-selection of the most responsive MTB, by genetic improvements of the bacteria, by reducing the feature sizes and the distance between conductors, by synchronizing with the motion of the MTB ramp-shaped current signals with multiplexing to neighbor conductors, and/or by inserting a high magnetic permeability layer just under the grid. Although the experiments were done with open-loop control, the use of sensors or imaging systems to detect the position of the microbeads being manipulated would allow closed-loop control and decision-making algorithms to be included in the computer software.

Experimental Setup

Each microfluidic channel with a width and a height of 50 μm×50 μm and a length of 4 cm and used for the initial experiments was engraved in glass (Schott Borofloat) with a thickness of 1.1 mm sandwiched with a similar glass and designed to prevent motions and fluctuations of the aqueous medium though serpentine designs at each ends. In order to obtain better images since observations in microfluidic channels showed significant aberrations in phase contrast mode, a drop of the solution of mixed MTB and microbeads was injected instead, using an injection syringe (Becton Dickinson ½ cc U 100 Insulin 28G), on a standard microscope slide such as the ones used for observation in biology and made of Fisher Scientific glass. Two cover slips 150 μm thick were deposited apart on the slide and a third one on the top in order to form an observation pool. To validate the feasibility of controlling the manipulation of microbeads by MTB from permanent magnets or computer software, the observation pool was placed on a custom-made electromagnetic grid made of 250 μm in diameter of parallel wires with a pitch of 500 μm arranged as two sets in parallel or oriented at right angle from each others. The grid was fixed to the x-y stage of a Zeiss Axiovert (www.zeiss.com) inverted microscope equipped with an acquisition CCD using Northern Eclipse software and set for observation in transmission (λ=530 nm) with phase contrast at 400×.

Computer-based Control

The static field circuit consisted of a power supply (by Agilent, model E3632A) sinking a constant current in one set of wires through a 1-ohm resistor. The varying field circuit for the other set of wires consisted of a power supply (by TTi, model TSX 1820P) sourcing a varying current through a 1-ohm resistor. The varying field circuit power supply was controlled via a GPIB interface card (by National Instrument, model PCI-GPIB+) connected to a PCI bus of a personal computer equipped with a Pentium III processor and running under Windows XP. The control software used to change the direction of the MTB in the observation pool was written in C++ and was executed by the Pentium processor. Control of the MTB was done by varying the field generated by the second set of wires by changing the amplitude and for some experiments the cycle time of the output voltage of the power supply.

Example of an Experimental Procedure

The first experiment was designed to evaluate the worst case scenario for directing the movement of bacteria for manipulation purpose, i.e. the response time of the MTB to rotate in an opposite direction. In this case, the electromagnetic grid with the two sets of wires arranged in parallel was used. The two sets were interleaved such that a wire with a static current was next to a wire with a varying opposite current. As such, the voltage across the 1-ohm resistor for the static field circuit was maintained constant at −7 volts DC (VDC) corresponding to a current of −7 A in this particular implementation, whereas a computer-controlled 50% duty cycle square wave varying between 0 VDC and 14 VDC corresponding to a varying current between 0 to +14 A was applied across the 1-ohm resistor for the varying field circuit. The waveforms were verified across the resistors with an oscilloscope (by Agilent, model 54641A) and recorded no significant signal distortions. These voltage levels were somewhat arbitrary chosen to provide a maximum magnetic field of 2 gauss (corresponding to a resulting current of 7 A) through the observation pool, which is small but sufficiently higher than the geomagnetic field but also adequate based on the previous response data from the bacteria (see FIG. 4B). The magnitude of the magnetic field at the observation pool was verified with previously calibrated magneto-resistive sensors (by Honeywell, model HMC1022) with signal conditioning and A/D conversion performed by a commercial acquisition card (by National Instrument, PCI-4472 8 channels dynamic signal acquisition board). The experimental results show that the frequency response of the MTB to rotate 180° was about 2 Hz (about 1 Hz for a 360° rotation).

Applications

Of course, the nature of the micro-object depends on the application of the method and system for controlling a micro-object according to the present invention, as will become more apparent upon reading the following non-restrictive brief descriptions of some applications thereof, as will also other objects, advantages and features of the present invention.

Bacterial Switch or Micro-switch

A “bacterial” switch or a micro-switch results from a small object capable of conducting electrical current being moved by magnetotactic bacteria using a method according to the present invention to connect or disconnect at least two electrical contacts.

Indeed, modifying the orientation of the magnetic field allows modifying the displacement path of MTB, thereby allowing controlling the path of the small conducting object during displacement thereof by the MTB so that the small object is selectively brought into contact or brought away from the electrical contacts, thereby acting as a micro-switch.

Bacterial Valve or Micro-valve

The basic principle of the “bacterial” valve is similar to bacterial switches, wherein a micro-object is moved by a MTB to open or close microfluidic flow or modify the direction of flow.

Bacterial Motor or Micro-motor

The magnetotactic bacteria can be used as actuators to move structures acting as linear (e.g. piston) or rotational micro-motors. The MTB are fixed to the structure to actuate, and a magnetic field is generated to orient the moving MTB along displacement path causing the MTB to act on the structures so as to produce the desired work.

Bacterial Memory

A bacterial memory according to a further aspect of the present invention can be created by moving a bacterium or a group of bacteria between two conductors or conducting pad and by “killing” selected bacteria to make permanent electrical connection through the chain of magnetosomes, the coating of conducting material on the bacteria. In this case, referring to FIG. 1, as will now appear obvious to a person skilled in the art, the magnetic field is generated to orient the MTB before the MTB is fixed to the objects, which, in this particular application, are the two conducting pads.

Autonomous Bacterial Microsystems (ABM)

According to another aspect of the present invention an autonomous bacterial microsystem (ABM) is provided where a microstructure is controlled by a method according to the present invention such as method 100, wherein the microstructure is propelled by magnetotactic bacteria. Such ABM can be though as an artificial organism.

The microstructure includes a wireless electronic circuit embedded onto the microstructure to remotely control the electromagnetic field to control the movement of MTB for movement around in a liquid. Sensors can be included to assist in the decision making process. Commands or behaviors of ABM can be pre-loaded or communicated by wireless channels.

Colony of ABM can be used to autonomously execute tasks where all ABM can be programmed with the same or various behaviors.

Bacterial Mixer or Micro-mixer

A “bacterial” mixer or micro-mixer results in micro-objects, being controlled using a method from the present inventions so as to move in various directions, in microfluidic systems, lab-on-a-chip, micro-wells, etc. to mix compounds. In some applications, the movement of the MTB alone may be sufficient to cause the mixing action.

An example of bacterial mixer 28 according to an illustrative embodiment of the present invention is illustrated in FIG. 8. The mixer 28 comprises first and second inlet reservoirs 30-32 and an outlet reservoir 34 in fluid communication with the two inlet reservoirs 30-32 and biased therefrom by a conduit 36. The mixer 28 further includes two facing rows of permanent magnets 38-40 on both sides of the conduit 36. Adjacent and facing magnets 38-40 have opposing polarities.

In operation, the two inlet reservoirs 30-32 comprise micro-scale liquids including MTB for mixing. The configuration of the magnets 38-40 causes the MTB to migrate towards the outlet reservoir in a non-linear fashion, thereby causing the mixing of the liquids.

Redundant Bacterial Micro-systems

Redundant bacterial micro-systems are systems where MTB change their route in the case of one or more failures. Beside redundancy for improving reliability, redundancy can be used for other tasks. For instance, a series of parallel fluidic micro-channels with different widths can be used to determine bead diameters or to go through a series of sensors. For instance, starting with the narrowest channel, if a bead being pushed by a MTB using the method 100 is blocked, the MTB pushing the bead will not reach a predetermined destination or location within a specific time period and then, the MTB can be re-routed to the next channels with a slightly larger width. The process can be repeated through successive channels with incremented width until the bead flow through the array of channels and reach the final destination. Of course, such application can be used with other objects than beads.

An exemple of such a redundant bacterial micro-system (RBMS) 42 is illustrated in FIG. 9. The RBMS 42 comprises two opposing reservoirs 44, micro-channels 46 made for example of siliconoxide or Pyrex™ glass for receiving traveling MTB, electromagnetic coils 48 buried under the micro-channels 46 at the intersection thereof, and electrical wires 50 also buried under the micro-channels, for generating the magnetic field allowing to orient the MTB a pre-determined displacement path.

Bacterial Sensors

Since several parameters such as toxicity, viscosity, temperature, etc., to name but a few can influence the displacement speed of MTB, by directing the motion of MTB and by measuring their travelling time, information related to the above-listed parameters and others can be obtained.

Bacterial Biosensors

According to a still another aspect of the present invention, a method for controlling micro-objects from the present invention can be used to create bacterial biosensors, where MTB are coated with a biological material or fixed to a micro-structure such as a microbead coated by antigens, phage, or other molecules, in a controlled fashion to sense or detect selected molecules or bio-organisms.

Bacterial In Vivo Actuators

Finally, a method and system for controlling micro-objects according to the present invention can also be used to conceive a bacterial in vivo actuator to deliver drugs or agents to tumour cells or other locations in the human body through blood vessels and capillaries using controlled MTB.

Although the present invention has been described hereinabove by way of preferred embodiments thereof, it can be modified, without departing from the spirit and nature of the subject invention as defined in the appended claims.

### REFERENCES

[1] Manz, A., Effenhauser, C. S., Burggraf, N., Harrison, D. J., Seiler, K. & Fluri, K. Electroosmotic pumping and electrophoretic separations for miniaturized chemical analysis systems. J. Micromech. Microeng. 4. 257, pages (1994).
[2] Pohl, H. A. Dielectrophoresis. Cambridge, UK: Cambridge University Press (1978).
[3] Schleifer, K. H., Schueler, D., Spring, S., Weizenegger, M., Amann, R., Ludwig, W. & Kohler, M. The genus Magnetospirillum, gen. nov., description of Magnetospirillum gryphiswaldense and transfer of Aquaspirillum magnetotacticum to Magnetospirillum magnetotacticum, comb. nov. Syst. Appl. Microbiol. 14, pages 379-385 (1991).
[4] Blackmore, R. P. Magnetotactic bacteria. Science. 190, pages 377-379 (1975).
[5] Happel, J. & Brenner, H. Low Reynolds number hydrodynamics. Martinus Nijhoff, The Hague, the Netherlands (1983).
[6] Darnton, N., Turner, L., Breuer, K. & Berg, H. C. Moving fluid with bacterial carpet. Biophysical Journal. 86, pages 1863-1870 (2004).
[7] Tung, S., Kim, J., Malshe, A., Lee, C. C. & Pooran, R. A cellular motor driven microfluidic system. Proc. of The 12th Int. Conf. on Solid State Sensors, Actuators and Microsystems (TRANSDUCERS '03), pages 678-681 (2003).
[8] Brown, D. A. & Berg, H. C. Temporal stimulation of chemotaxis in Escherichia coli. Proc. Natl. Acad. Sci. USA. 71, pages 1388-1392 (1974).
[9] Berg, H. C. & Brown, D. A. Chemotaxis in Escherichia coli analyzed by three-dimensional tracking. Nature. 239, pages 500-504 (1972).
[10] Ford, R. M., Phillips, B. R., Quinn, J. A. & Lauffenburger, D. A. Measurement of bacterial random motility and chemotaxis coefficients. I. Stopped-flow diffusion chamber assay. Biotech. and Bioeng. 37(7), pages 647-660 (1991).
[11] Armitage, J. P. Bacterial motility and chemotaxis. Sci. Progr. 76, pages 451-477 (1992).
[12] Frankel, R. B. & Blakemore, R. P. Navigational compass in magnetic bacteria. J. I of Magn. and Magn. Materials. 15-18(Part 3), pages 1562-1564 (1980).
[13] Denham, C., Blakemore, R. & Frankel, R. Bulk magnetic properties of magnetotactic bacteria. IEEE Trans. on Magnetics. 16(5), pages 1006-1007 (1980).
[14] Debarros, H., Esquivel, D. M. S. & Farina M. Magnetotaxis. Sci. Progr. 74, pages 347-359 (1990).
[15] Bahaj, A. S., James, P. A. B., and Moeschler, F. D. Low magnetic-field separation system for metal-loaded magnetotactic bacteria. J. of Magn. and Magn. Materials. 177-181(Part 2), pages 14531454 (1998).
[16] Lee, H., Purdon, A. M., Chu, V. & Westervelt, R. M. Controlled assembly of magnetic nanoparticles from magnetotactic bacteria using microelectromagnets arrays. Nano Lett. 4(5), pages 995-998 (2004).
[17] Lee, H., Purdon, A. M., Chu, V. & Westervelt, R. M. Micromanipulation of biological systems with microelectromagnets. IEEE Trans. on Magnetics. 40(4), pages 2991-2993 (2004).
[18] Heyen, U. & Schuler, D. Growth and magnetosome formation by microaerophilic Magnetospirillum strains in an oxygen-controlled fermentor. Appl. Microbiol. Biotechnol. 61, pages 536-544 (2003).
[19] Blakemore, R. P. Magnetotactic bacteria. Annu. Rev. Microbiol. 36, pages 217-238 (1982).
[20] Seong, S. & Park, T. H. Swimming characteristics of magnetic bacterium, Magnetospirillum sp. AMB-1, and implications as toxicity measurement. Biotech. and Bioeng. 76(1), pages 11-16 (2001).
[21] Deng, T., Whitesides, G. M., Radhakrishnan, M., Zabow, G. & Prentiss, M. Manipulation of magnetic microbeads in suspension using micromagnetic systems fabricated with soft lithography. Appl. Phys. Left. 78(12), pages 1775-1777 (2001).

What is claimed is:

1. A method for controlling at least one micro-object comprising:
providing at least one magnetotactic bacterium (MTB); said at least one magnetotactic bacterium being self-propulsive;
coupling the at least one micro-object with said at least one magnetotactic bacterium, causing the at least one micro-object to move in unison with said at least one magnetotactic bacterium; and
generating a magnetic field for orienting said at least one magnetotactic bacterium along a displacement path;
whereby, in operation, modifying the orientation of said magnetic field allows modifying said displacement path of said at least one magnetotactic bacterium, thereby allowing controlling the path of the at least one micro-object during displacement thereof by said at least one magnetotactic bacterium.

2. A method as recited in claim 1, wherein said magnetic field is created using a direct current (DC) signal.

3. A method as recited in claim 1, wherein said magnetic field is slightly greater than Earth magnetic field.

4. A method as recited in claim 1, wherein said magnetic field is a low energy DC magnetic field.

5. A method as recited in claim 1, wherein coupling the at least one micro-object with said at least one magnetotactic bacterium includes fixing said at least one magnetotactic bacterium to the at least one micro-object.

6. A method as recited in claim 5, fixing said at least one magnetotactic bacterium to the at least one micro-object includes forcing said at least one MTB to generate activation of lipopolysaccharides, causing the at least one micro-object to stick to said at least one MTB when said at least one micro-object comes into contact with said at least one MTB.

7. A method as recited in claim 1, wherein providing at least one magnetotactic bacterium includes providing an active solution of highly concentrated magnetospirillum gryphiswaldense bacteria or of magnetospirillum magnetotacticum bacteria.

8. A method as recited in claim 1, wherein said at least one MTB includes a plurality of MTB.

9. A method as recited in claim 1, wherein said at least one micro-object includes a plurality of micro-objects.

10. A method as recited in claim 1, wherein said at least one micro-object includes at least one microbead.

11. A method as recited in claim 1, wherein said at least one micro-object is capable of conducting electrical current;
whereby, in operation, modifying the orientation of said magnetic field allows modifying said displacement path of said at least one magnetotactic bacterium, thereby allowing controlling the path of the at least one micro-object during displacement thereof by said at least one magnetotactic bacterium so that said at least one micro-object is selectively brought into contact or brought away from two electrical contacts, thereby acting as a micro-switch.

12. A method as recited in claim 1, wherein the at least one micro-object is controlled to open or close microfluidic flow or modify a direction of microfluidic flow.

13. A method as recited in claim 1, wherein the at least one micro-object includes a micro-piston;
whereby, in operation, said orientation of said magnetic field is first modified to bring said at least one MTB into contact with the micro-piston; said micro-piston being then coupled with said at least one MTB; then modifying the orientation of said magnetic field allows modifying said displacement path of said at least one magnetotactic bacterium, thereby allowing pushing or pulling the micro-piston.

14. A method as recited in claim 1, wherein the at least one micro-object is a microstructure including a wireless electronic circuit embedded onto said microstructure to remotely control said electromagnetic field.

15. A method as recited in claim 14, wherein remote control of said electromagnetic field by said wireless electronic structure is achieved by commands pre-loaded into said electronic circuit or communicated through a wireless channel.

16. A system for controlling at least one micro-object comprising:
at least one magnetotactic bacterium (MTB) for coupling with the at least one micro-object for movement in unison; said at least one magnetotactic bacterium being self-propulsive; and
a magnetic field generator for orienting said at least one magnetotactic bacterium along a displacement path;
whereby, in operation, modifying the orientation of said magnetic field allows modifying said displacement path of said at least one magnetotactic bacterium, thereby allowing controlling the path of the at least one micro-object during displacement thereof by said at least one magnetotactic bacterium.

17. A system as recited in claim 16, wherein said magnetic field generator includes at least two pairs of conductors for receiving four independent electrical signals; said two pairs of conductors being configured in two generally perpendicular facing pairs so as to generally define a rectangle to enclose said at least one MTB and the at least one micro-object;
whereby, in operation, modulating signal amplitudes in adjacent pairs of conductors among said at least two pairs of conductors yields a desired location of an artificial pole for orienting said at least one MTB.

18. A system as recited in claim 17, wherein said magnetic field generator includes a grid of conductors; said grid including two sets of conductors generally in parallel passing at right angle from each other.

19. A system as recited in claim 18, wherein the at least one micro-object includes a plurality of micro-objects and said at least one MTB includes a plurality of MTB; the system further comprising a controller for coordinating relative displacements of at least some of said plurality of micro-objects in unison with respective MTB so as to be moved at selected positions on said grid so for displaying information therethrough.

20. A system as recited in claim 17, further comprising a signal controller coupled to each said conductors.

21. A system as recited in claim 20, further comprising an input device and a sensor both coupled to said signal controller to allow a user control and feedback of said at least one MTB.

22. A system as recited in claim 21, wherein said sensor includes at least one of a photovoltaic cell, a magnetic sensor, and a chemical sensor.

23. A system as recited in claim 16, wherein said magnetic field generator allows creating a low energy DC magnetic field.

24. A system as recited in claim 16, wherein said magnetic field generator includes at least one permanent magnet.

25. A system as recited in claim 16, further comprising coupling means for mounting the at least one micro-object to said at least one MTB.

26. A system as recited in claim 25, wherein said coupling means includes an adhesive between said at least one MTB and the at least one micro-object for fixing said at least one MTB to the at least one micro-object.

27. A system as recited in claim 16, further comprising a controller coupled to said magnetic field generator for controlling the orientation of said magnetic field.

28. A system as recited in claim 27, wherein said controller is in the form of an embedded controller secured to said at least one magnetotactic bacteria; said system further comprising an integrated sensor coupled to said embedded controller allowing said at least one magnetotactic bacteria-based system changing its direction of motion according to new occurring conditions as detected by said integrated sensor.

29. A system as recited in claim 16, wherein said at least one MTB includes a plurality of MTB.

30. A system as recited in claim 16, wherein said at least one micro-object includes a plurality of micro-objects.

31. A system as recited in claim 16, wherein said at least one micro-object includes at least one microbead.

32. A method for controlling at least one magnetotactic bacterium (MTB), said at least one MTB being self-propulsive along a displacement path, the method comprising:
generating a magnetic field so as to effect the at least one MTB; said magnetic field being characterized by a pole; said magnetic field affecting said at least one MTB by biasing the displacement path towards said pole; and
selectively modifying the displacement path of the at least one MTB by modifying said pole of said magnetic field.

33. A method as recited in claim 32, wherein selectively modifying the path of the at least one MTB by modifying said pole of said magnetic field brings at least one of said at least one MTB between a respective pair of conductors; said method further comprising: killing at least one selected from said at least one MTB, resulting in at least one corresponding permanent connection between said respective pair of conductors.

34. A method as recited in claim 32, further comprising:
fixing the at least one MTB to a structure to be actuated; whereby, in operation said magnetic field is generated to orient the at least one MTB along a displacement path causing the at least one MTB to act on said structure so as to cause the actuation thereof.

35. A method as recited in claim 34, wherein said structure is a piston or a micro-motor.

36. A method as recited in claim 32, for mixing a fluid, said method further comprising:
providing the fluid in a container with the at least one MTB; and
frequently modifying the displacement path of the at least one MTB yielding a mixing action on the fluid.

37. A method as recited in claim 32, for characterizing a fluid, said method further comprising:
providing the fluid in a container with the at least one MTB;
generating said magnetic field so as to cause said at least one MTB to travel from a known path, yielding a travelling time; and
comparing said travelling time to a predetermined travelling time resulting from similar MTB travelling in a characterized fluid to determine at least one unknown characteristic of said fluid.
